(19) United States
(12) Patent Application Publication
(10) Pub. No.: US 2010/0210765 A1
(43) Pub. Date: Aug. 19, 2010

(54) RESIST UNDERLAYER FILM-FORMING COMPOSITION, PRODUCTION METHOD OF SEMICONDUCTOR DEVICE USING THE SAME, AND ADDITIVE FOR RESIST UNDERLAYER FILM-FORMING COMPOSITION

(75) Inventors: Makoto Nakajima, Toyama-shi (JP); Hideo Suzuki, Funabashi-shi (JP)

Correspondence Address: OLIFF & BERRIDGE, PLC, P.O. BOX 320850, ALEXANDRIA, VA, 22320-4850, US

(73) Assignee: NISSAN CHEMICAL INDUSTRIES, LTD., Tokyo (JP)

(21) Appl. No.: 12/678,311

(22) PCT Filed: Sep. 30, 2008
(86) PCT No.: PCT/JP2008/067758

§ 371 Date: Mar. 16, 2010

(30) Foreign Application Priority Data
Oct. 1, 2007 (JP) 2007-257719

(51) Int. Cl. C08K 5/1515; C08K 5/092; C07D 303/02

(52) U.S. Cl. 524/114; 524/285; 549/544

(57) ABSTRACT

There is provided a composition for forming a resist underlayer film that the adhesion with a resist applied on the resist underlayer film is enhanced and the collapse of a resist pattern is suppressed. A resist underlayer film-forming composition for lithography comprising: a polymer having silicon atoms in the backbone; a compound of a polycyclic structure; and an organic solvent, wherein the compound of a polycyclic structure has at least two carboxyl groups as substituents; the two carboxyl groups are individually bonded to two carbon atoms adjacent to each other forming the polycyclic structure; and the two carboxyl groups both have an endo configuration or an exo configuration, or have a cis configuration.

### TECHNICAL FIELD

The present invention relates to a composition containing a polymer having silicon atoms in the backbone, a compound of a specific polycyclic structure and an organic solvent, for forming a resist underlayer film provided between a substrate and a resist by applying the composition on the substrate and curing the composition. The present invention also relates to a compound of a specific polycyclic structure contained in the composition.

### BACKGROUND ART

There is disclosed a coating liquid for forming a coating film containing a compound having a silanol group obtained by hydrolyzing at least two types of specific alkoxysilane compounds in the presence of water and a catalyst in a specific organic solvent (see Patent Document 1). Two silanol groups are condensed to form a polymer having silicon atoms in the backbone.

In addition, there is disclosed that a cured film of an organopolysiloxane is used as a resist underlayer film formed on a semiconductor substrate (see Patent Document 2). The cured film is to be dry-etched using a photoresist pattern as a mask and using CF₄ gas. Thereafter, during the removal of the photoresist pattern, the cured film can remain.

Further, there is disclosed a pattern forming method including: forming an organic film on a substrate as a resist underlayer film; forming an inorganic film containing silicon atoms on the resist underlayer film as a first resist intermediate layer film; forming a silicon resin film containing a silicon resin on the first resist intermediate layer film as a second resist intermediate layer film; forming a photoresist film on the second resist intermediate layer film and subjecting the photoresist film to exposure and development to form a resist pattern; etching the first and second resist intermediate layer films using the resist pattern as a mask; etching the resist underlayer film using the first and second resist intermediate layer films after the etching as masks; and etching the substrate using the resist underlayer film after the etching as a mask (see Patent Document 3).

### [Patent Document 1]

Japanese Patent Application Publication No. JP-A-3-045510

### [Patent Document 2]

Japanese Patent Application Publication No. JP-A-2-103052

### [Patent Document 3]

Japanese Patent Application Publication No. JP-A-2007-47580

### DISCLOSURE OF THE INVENTION

### Problem to be Solved by the Invention

A resist underlayer film containing silicon tends to have poor adhesion to an organic resist. Therefore, when a resist pattern is attempted to be formed on the resist underlayer film, there is such a problem that a collapse of the resist pattern occurs frequently. The “organic resist” is defined in the present specification as a positive or negative resist containing no silicon resin such as polysiloxane and polysilane.

In recent years, according to the miniaturization and the high-integration of semiconductor elements, the miniaturization of resist patterns has been progressing. As the width of a resist pattern is decreased, the resist pattern collapses more easily. Therefore, countermeasures for the problem have become furthermore important.

### Means for Solving the Problem

The present invention is based on a concept that by blending a compound of a specific polycyclic structure in a resist underlayer film-forming composition, the adhesion between a resist underlayer film formed from the composition and a resist can be enhanced and the collapse of a resist pattern can be suppressed. It is satisfactory that the compound of a specific polycyclic structure is contained finally in the resist underlayer film-forming composition, and the stage of the production process in which the compound is blended is not limited. When two or more layers of resist underlayer films are formed between a substrate and a resist, a resist underlayer film directly under the resist may be expressed as a resist intermediate layer film.

The present invention relates to a resist underlayer film-forming composition for lithography containing a polymer having silicon atoms in the backbone, a compound of a polycyclic structure and an organic solvent in which: the compound of a polycyclic structure has at least two carboxyl groups as substituents; the two carboxyl groups are individually bonded to two carbon atoms adjacent to each other forming the polycyclic structure, and the two carboxyl groups both have an endo configuration or an exo configuration, or have a cis configuration.

The compound of a polycyclic structure is a compound of Formula (1):

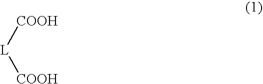

(where L is derived from a hydrocarbon of a polycyclic structure of Formula (2), Formula (3), Formula (4), Formula (5), Formula (6) or Formula (7):

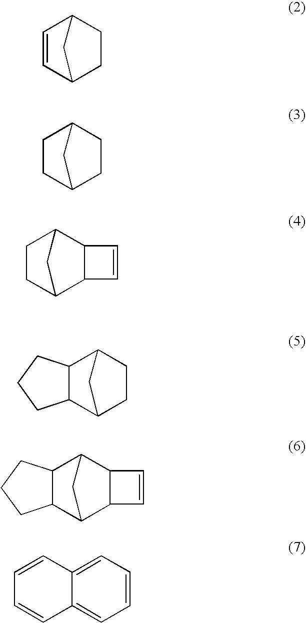

and is a divalent group in which two carboxyl groups are bonded to two carbon atoms adjacent to each other forming the polycyclic structure; and the hydrocarbon of a polycyclic structure of Formula (2) to Formula (7) can further have a substituent and/or can be epoxidized).

In the hydrocarbon of a polycyclic structure of Formula (2) to Formula (7), at least one hydrogen atom may be replaced by a halogen atom. Examples of the hydrogen atom include fluorine, chlorine, bromine and iodine.

The two carboxyl groups both have an endo configuration or an exo configuration, or have a cis configuration. It can be also expressed that the two carboxyl groups exist in the same plane. In other words, the configuration of the two carboxyl groups does not take a configuration in which one of the two has an endo configuration and the other has an exo configuration, and a trans configuration.

Examples of the compound of a polycyclic structure include alicyclic hydrocarbons having a bicyclo ring, a tricyclo ring or a tetracyclo ring.

Examples of the compound of a polycyclic structure include alicyclic dicarboxylic acids of a polycyclic structure.

Examples of the compound of a polycyclic structure include 3,4-epoxytetracyclo[5.4.1.0^{2,6}.0^{8,11}]dodeca-9-ene-9,10-dicarboxylic acid.

Examples of the polymer having silicon atoms in the backbone contained in the resist underlayer film-forming composition of the present invention include hydrolysis-condensates of at least two types of alkoxysilanes. Here, the shape of the backbone is not limited to a linear chain and includes also a branched chain and a network chain. Examples of the polymer include a polysiloxane.

In addition, another aspect of the present invention relates to an additive for a resist underlayer film-forming composition containing a compound of a polycyclic structure having at least two carboxyl groups as substituents in which the two carboxyl groups are individually bonded to two carbon atoms adjacent to each other forming a polycyclic structure and the two carboxyl groups both have an endo configuration or an exo configuration, or have a cis configuration.

Here, examples of the compound of a polycyclic structure include 3,4-epoxytetracyclo[5.4.1.0^{2,6}.0^{8,11}]dodeca-9-ene-9,10-dicarboxylic acid.

### EFFECTS OF THE INVENTION

The resist underlayer film-forming composition of the present invention can suppress the collapse of a resist pattern on a resist underlayer film formed from the composition by containing a compound of a specific polycyclic structure, that is, a compound of a polycyclic structure having at least two carboxyl groups as substituents in which the two carboxyl groups are individually bonded to two carbon atoms adjacent to each other forming the polycyclic structure and the two carboxyl groups both have an endo configuration or an exo configuration, or have a cis configuration. In other words, the compound of a specific polycyclic structure is useful as an additive for a resist underlayer film-forming composition.

The two carboxyl groups both have an endo configuration or an exo configuration, or have a cis configuration and the two carboxyl groups are adjacent to each other. Therefore, it is considered that when the resist underlayer film-forming composition of the present invention is applied and then, thermally cured, water is generated by a dehydration reaction between the carboxyl groups and the generated water accelerates a hydrolysis and a condensation reaction of alkoxysilanes remaining in the composition. As a result thereof, the formed resist underlayer film becomes rigid. On the other hand, when the configuration of the two carboxyl groups take a configuration in which one of the two is an endo configuration and the other is an exo configuration, or a trans configuration, the two carboxyl groups are apart from each other, so that a dehydration reaction is unlikely to be effected.

Further, a compound of a polycyclic structure exhibits hydrophobicity and is easily concentrated on the surface of the film, so that it is inferred that the compound enhances the adhesion of the resist underlayer film to an organic resist formed on the film. Particularly, when the organic resist, too, contains a compound of a polycyclic structure, it is considered that an enhancing effect of the adhesion of the organic resist to the resist underlayer film containing a compound of a polycyclic structure on the surface thereof is high. Examples of the compound of a polycyclic structure contained in the organic resist include adamantane and derivatives thereof.

### BEST MODES FOR CARRYING OUT THE INVENTION

Specific examples of the compound of a polycyclic structure that is contained in the resist underlayer film-forming composition of the present invention and in which the compound of a polycyclic structure has at least two carboxyl groups as substituents in which the two carboxyl groups are individually bonded to two carbon atoms adjacent to each other forming the polycyclic structure and the two carboxyl groups both have an endo configuration or an exo configuration, or have a cis configuration are shown in Formula (8) to Formula (15). However, the compound of a polycyclic structure is not limited to these specific examples.

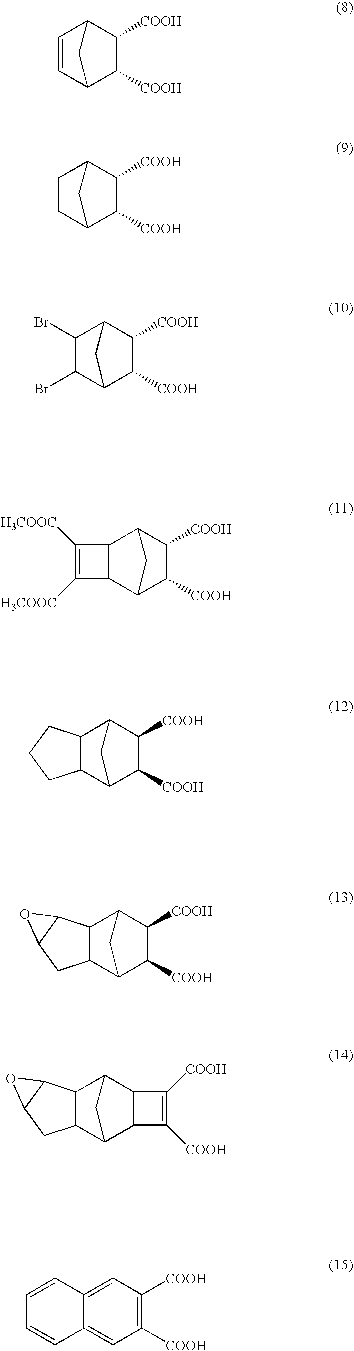

The organic solvent contained in the resist underlayer film-forming composition of the present invention is preferably an organic solvent capable of dissolving the compound of a polycyclic structure. Specific examples of the organic solvent include ethanol, propylene glycol monomethyl ether, propylene glycol monopropyl ether, propylene glycol monomethyl ether acetate, ethyl lactate, cyclohexanone and γ-butyrolactone.

When a component remaining after removing the organic solvent from the resist underlayer film-forming composition of the present invention is defined as the solid content, the compound of a polycyclic structure is contained in an amount of, for example 0.1% by mass to 30% by mass, or 1% by mass to 20% by mass, based on the mass of the solid content. The solid content is contained in an amount of, for example 0.1% by mass to 30% by mass, or 1% by mass to 15% by mass, based on the mass of the resist underlayer film-forming composition of the present invention. The polymer having silicon atoms in the backbone is contained in an amount of, for example 70% by mass to 99.9% by mass, or 85% by mass to 99% by mass, based on the mass of the solid content.

Further, to the resist underlayer film-forming composition of the present invention, water may be added. By adding water, stability of the resist underlayer film-forming composition of the present invention can be enhanced. In this case, water may be contained in an amount of, for example 5% by mass to 20% by mass, based on the mass of the composition (solution).

The resist underlayer film-forming composition of the present invention may further contain an acid generator in an amount of, for example 0.1% by mass to 20% by mass or less, based on the mass of the solid content. Examples of such an additive include onium salts such as sulfonium salts, benzothiazolium salts, ammonium salts, iodonium salts and phosphonium salts. The acid generator is classified into a thermo-acid generator generating an acid by a thermal decomposition thereof and a photo-acid generator generating an acid by a light irradiation. For example, sulfonium salts and iodonium salts have characteristics as a photo-acid generator, however, may also have characteristics as a thermo-acid generator.

Quaternary ammonium salts and quaternary phosphonium salts are preferably used for accelerating the crosslinking reaction of a polymer after a composition containing no crosslinker is applied and then, is cured. Examples of the quaternary ammonium salt include benzyltriethylammonium chloride, benzyltrimethylammonium chloride, benzyltributylammonium chloride, tetramethylammonium chloride, tetraethylammonium bromide, tetraethylammonium chloride, tetrapropylammonium bromide, tetrabutylammonium bromide, tributylmethylammonium chloride, trioctylmethylammonium chloride and phenyltrimethylammonium chloride, and for example, benzyltriethylammonium chloride is selected. Examples of the quaternary phosphonium salt include ethyltriphenylphosphonium bromide, ethyltriphenylphosphonium iodide, benzyltriphenylphosphonium chloride, butyltriphenylphosphonium bromide and tetrabutylphosphonium bromide, and for example, ethyltriphenylphosphonium bromide or tetrabutylphosphonium bromide is selected. The quaternary ammonium salt or the quaternary phosphonium salt may be contained in the composition in an amount of, for example 0.001% by mass to 10% by mass, or 0.01% by mass to 5% by mass, based on the mass of the solid content.

The resist underlayer film-forming composition of the present invention may contain a surfactant in an amount of, for example 0.01% by mass to 2% by mass, based on the mass of the solid content. The surfactant can enhance the applicability of the composition to the substrate and examples thereof include nonionic surfactants and fluorinated surfactants.

A using example of the resist underlayer film-forming composition of the present invention is as follows. An organic film (first resist underlayer film) is formed on a substrate such as a silicon wafer, and on the organic film, the resist underlayer film-forming composition of the present invention is applied, followed by curing the composition by heating or the like to form a resist underlayer film (second resist underlayer film). On this resist underlayer film, an organic resist layer is formed and the organic resist layer is subjected to exposure, post exposure bake (abbreviated as PEB) if necessary, and development to form a resist pattern. Using the formed resist pattern as a mask, the resist underlayer film is dry-etched and further, the organic film on the substrate is dry-etched. Then, when the resist pattern remains after the dry-etching, it is removed. On the substrate, an insulating film such as an oxide film, a semiconductor film such as a polysilicon or a conductive film may be formed.

The resist underlayer film-forming composition of the present invention is applied to the lithography process in a production process of: semiconductor elements (diodes, transistors, memory and the like) using a semiconductor substrate or a compound semiconductor substrate such as a silicon wafer, gallium arsenide and gallium phosphide, and an insulating substrate such as a glass substrate and a plastic substrate; and electronic equipment (mobile phones, television sets, personal computers and the like) equipped with the semiconductor element. In the present specification, a semiconductor element and an electronic equipment equipped with the same are defined as the semiconductor device.

Specific examples of the alkoxysilane that is a raw material monomer of a polymer having silicon atoms in the backbone include tetramethoxysilane, tetraethoxysilane, tetra(n-propoxy)silane, tetra(iso-propoxy)silane, tetra(n-butoxy)silane, tetra(iso-butoxy)silane, tetra(sec-butoxy)silane, tetra(tert-butoxy)silane, methyltrimethoxysilane, methyltriethoxysilane, methyltri(n-propoxy)silane, methyltri(iso-propoxy)silane, methyltri(n-butoxy)silane, methyltri(iso-butoxy)silane, methyltri(sec-butoxy)silane, methyltri(tert-butoxy)silane, ethyltrimethoxysilane, ethyltriethoxysilane, ethyltri(n-propoxy)silane, ethyltri(iso-propoxy)silane, ethyltri(n-butoxy)silane, ethyltri(iso-butoxy)silane, ethyltri(sec-butoxy)silane, ethyltri(tert-butoxy)silane, n-propyltrimethoxysilane, n-propyltriethoxysilane, n-propyltri(n-propoxy)silane, n-propyltri(iso-propoxy)silane, n-propyltri(n-butoxy)silane, n-propyltri(iso-butoxy)silane, n-propyltri(sec-butoxy)silane, n-propyltri(tert-butoxy)silane, iso-propyltrimethoxysilane, iso-propyltriethoxysilane, iso-propyltri(n-propoxy)silane, iso-propyltri(iso-propoxy)silane, iso-propyltri(n-butoxy)silane, iso-propyltri(iso-butoxy)silane, iso-propyltri(sec-butoxy)silane, iso-propyltri(tert-butoxy)silane, n-butyltrimethoxysilane, n-butyltriethoxysilane, n-butyltri(n-propoxy)silane, n-butyltri(iso-propoxy)silane, n-butyltri(n-butoxy)silane, n-butyltri(iso-butoxy)silane, n-butyltri(seq-butoxy)silane, n-butyltri(tert-butoxy)silane, sec-butyltrimethoxysilane, sec-butyltriethoxysilane, sec-butyltri(n-propoxy)silane, sec-butyltri(iso-propoxy)silane, sec-butyltri(n-butoxy)silane, sec-butyltri(iso-butoxy)silane, sec-butyltri(sec-butoxy)silane, sec-butyltri(tert-butoxy)silane, tert-butyltrimethoxysilane, tert-butyltriethoxysilane, tert-butyltri(n-propoxy)silane, tert-butyltri(iso-propoxy)silane, tert-butyltri(n-butoxy)silane, tert-butyltri(iso-butoxy)silane, tert-butyltri(sec-butoxy)silane, tert-butyltri(tert-butoxy)silane, vinyltrimethoxysilane, vinyltriethoxysilane, vinyltri(n-propoxy)silane, vinyltri(iso-propoxy)silane, vinyltri(n-butoxy)silane, vinyltri(iso-butoxy)silane, vinyltri(sec-butoxy)silane, vinyltri(tert-butoxy)silane, phenyltrimethoxysilane, phenyltriethoxysilane, phenyltri(n-propoxy)silane, phenyltri(iso-propoxy)silane, phenyltri(n-butoxy)silane, phenyltri(iso-butoxy)silane, phenyltri(sec-butoxy)silane, phenyltri(tert-butoxy)silane, dimethyldimethoxysilane, dimethyldiethoxysilane, dimethyldi(n-propoxy)silane, dimethyldi(iso-propoxy)silane, dimethyldi(n-butoxy)silane, dimethyldi(iso-butoxy)si lane, dimethyldi(sec-butoxy)silane, dimethyldi(tert-butoxy)silane, diethyldimethoxysilane, diethyldiethoxysilane, diethyldi(n-propoxy)silane, diethyldi(iso-propoxy)silane, diethyldi(n-butoxy)silane, diethyldi(iso-butoxy)silane, diethyldi(sec-butoxy)silane, diethyldi(tert-butoxy)silane, di(n-propyl)dimethoxysilane, di(n-propyl)diethoxysilane, di(n-propyl)di(n-propoxy)silane, di(n-propyl)di(iso-propoxy)silane, di(n-propyl)di(n-butoxy)silane, di(n-propyl)di(iso-butoxy)silane, di(n-propyl)di(sec-butoxy)silane, di(n-propyl)di(tert-butoxy)silane, di(iso-propyl)dimethoxysilane, di(iso-propyl)diethoxysilane, di(iso-propyl)di(n-propoxy)silane, di(iso-propyl)di(iso-propoxy)silane, di(iso-propyl)di(n-butoxy)silane, di(iso-propyl)di(iso-butoxy)silane, di(iso-propyl)di(sec-butoxy)silane, di(iso-propyl)di(tert-butoxy)silane, di(n-butyl)dimethoxysilane, di(n-butyl)diethoxysilane, di(n-butyl)di(n-propoxy)silane, di(n-butyl)di(iso-propoxy)silane, di(n-butyl)di(n-butoxy)silane, di(n-butyl)di(iso-butoxy)silane, di(n-butyl)di(sec-butoxy)silane, di(n-butyl)di(tert-butoxy)silane, di(iso-butyl)dimethoxysilane, di(iso-butyl)diethoxysilane, di(iso-butyl)di(n-propoxy)silane, di(iso-butyl)di(iso-propoxy)silane, di(iso-butyl)di(n-butoxy)silane, di(iso-butyl)di(iso-butoxy)silane, di(iso-butyl)di(sec-butoxy)silane, di(iso-butyl)di(tert-butoxy)silane, di(sec-butyl)dimethoxysilane, di(sec-butyl)diethoxysilane, di(sec-butyl)di(n-propoxy)silane, di(sec-butyl)di(iso-propoxy)silane, di(sec-butyl)di(n-butoxy)silane, di(sec-butyl)di(iso-butoxy)silane, di(sec-butyl)di(sec-butoxy)silane, di(sec-butyl)di(tert-butoxy)silane, di(tert-butyl)dimethoxysilane, di(tert-butyl)diethoxysilane, di(tert-butyl)di(n-propoxy)silane, di(tert-butyl)di(iso-propoxy)silane, di(tert-butyl)di(n-butoxy)silane, di(tert-butyl)di(iso-butoxy)silane, di(tert-butyl)di(sec-butoxy)silane, di(tert-butyl)di(tert-butoxy)silane, divinyldimethoxysilane, divinyldiethoxysilane, divinyldi(n-propoxy)silane, divinyldi(iso-propoxy)silane, divinyldi(n-butoxy)silane, divinyldi(iso-butoxy)silane, divinyldi(sec-butoxy)silane, divinyldi(tert-butoxy)silane, diphenyldimethoxysilane, diphenyldiethoxysilane, diphenyldi(n-propoxy)silane, diphenyldi(iso-propoxy)silane, diphenyldi(n-butoxy)silane, diphenyldi(iso-butoxy)silane, diphenyldi(sec-butoxy)silane and diphenyldi(tert-butoxy)silane.

From the above alkoxysilanes, for example tetraethoxysilane, phenyltrimethoxysilane, vinyltrimethoxysilane and methyltriethoxysilane can be selected.

As a catalyst for accelerating the hydrolysis (and condensation reaction) of an alkoxysilane, an acid dissolved in water or an organic solvent can be used. Examples of such an acid include: inorganic acids such as hydrochloric acid, nitric acid, phosphoric acid and sulfuric acid; and organic acids such as sulfonic acid, p-toluenesulfonic acid, benzenesulfonic acid, formic acid, acetic acid and propionic acid. As such a catalyst, there may be used a compound of a polycyclic structure having at least two carboxyl groups as substituents, for example an alicyclic dicarboxylic acid of a polycyclic structure. Particularly, when the two carboxyl groups are individually bonded to carbon atoms adjacent to each other forming a polycyclic structure and the two carboxyl groups both have an endo configuration or an exo configuration, or have a cis configuration, the compound of a polycyclic structure is not necessary to be separately blended in the resist underlayer film-forming composition of the present invention.

Hereinafter, the present invention is described more specifically referring to Synthetic Examples and Examples, which should not be construed as limiting the scope of the present invention. Here, as the alicyclic dicarboxylic acid of a polycyclic structure used in the following Synthetic Examples and Examples, one that is purified in order to remove impurities (particularly metals) can be used, if necessary.

### EXAMPLES

In the present specification, the following average molecular weight of polymers is a measurement result by gel permeation chromatography (hereinafter, abbreviated as GPC). The used apparatus, conditions and the like are as follows.

GPC apparatus: HLC-8220 GPC (trade name; manufactured by Tosoh Corporation)
GPC column: Shodex (registered trade mark) KF803L, KF802, KF8O₁ (trade names; manufactured by Showa Denko K.K.)
Column temperature: 40° C.
Solvent: tetrahydrofuran (THF)
Flow rate: 1.0 mL/min
Standard sample: polystyrene (manufactured by Showa Denko K.K.)

### Synthesis Example 1

In the present Synthetic Example, as an alkoxysilane, tetraethoxysilane, phenyltrimethoxysilane, vinyltrimethoxysilane and methyltriethoxysilane (each manufactured by Tokyo Chemical Industry Co., Ltd.) were applied as a raw material monomer.

62.47 g of tetraethoxysilane, 8.49 g of phenyltrimethoxysilane, 6.35 g of vinyltrimethoxysilane, 7.64 g of methyltriethoxysilane and 84.95 g of ethanol were charged into a 300 mL flask to dissolve them and the resultant mixed solution was warmed and refluxed while stirring the solution with a magnetic stirrer. Next, an aqueous solution in which 1.56 g of hydrochloric acid was dissolved in 28.55 g of ion-exchanged water was added to the mixed solution. The reaction was effected for 2 hours and the resultant reaction solution was cooled down to a room temperature. After the completion of the reaction, as by-products, ethanol and methanol were generated.

Thereafter, 200 g of propylene glycol monomethyl ether acetate was added to the reaction solution, and ethanol, methanol, water and hydrochloric acid were distilled off under reduced pressure to obtain a solution containing a hydrolysis condensate (polymer). The molecular weight of the polymer obtained according to the present Synthetic Example measured by GPC was 6,000 of weight average molecular weight Mw converted into that of polystyrene.

### Synthesis Example 2

In the present Synthetic Example, as an alkoxysilane, tetraethoxysilane, phenyltrimethoxysilane and vinyltrimethoxysilane (each manufactured by Tokyo Chemical Industry Co., Ltd.) were applied as a raw material monomer.

63.28 g of tetraethoxysilane, 8.60 g of phenyltrimethoxysilane, 12.86 g of vinyltrimethoxysilane and 84.75 g of ethanol were charged into a 300 mL flask to dissolve them and the resultant mixed solution was warmed and refluxed while stirring the solution with a magnetic stirrer. Next, an aqueous solution in which 1.58 g of hydrochloric acid was dissolved in 28.92 g of ion-exchanged water was added to the mixed solution. The reaction was effected for 2 hours and the resultant reaction solution was cooled down to a room temperature. After the completion of the reaction, as by-products, ethanol and methanol were generated.

Thereafter, 200 g of propylene glycol monomethyl ether acetate was added to the reaction solution, and ethanol, methanol, water and hydrochloric acid were distilled off under reduced pressure to obtain a solution containing a hydrolysis condensate (polymer). The molecular weight of the polymer obtained according to the present Synthetic Example measured by GPC was 4,400 of weight average molecular weight Mw converted into that of polystyrene.

### Synthesis Example 3

In the present Synthetic Example, as an alkoxysilane, tetraethoxysilane, phenyltrimethoxysilane and methyltriethoxysilane (each manufactured by Tokyo Chemical Industry Co., Ltd.) were applied as a raw material monomer.

31.53 g of tetraethoxysilane, 2.50 g of phenyltrimethoxysilane, 15.74 g of methyltriethoxysilane and 66.59 g of ethanol were charged into a 300 mL flask to dissolve them and the resultant mixed solution was warmed and refluxed while stirring the solution with a magnetic stirrer. Next, a solution in which 0.92 g of cis-5-norbornene-endo-2,3-dicarboxylic acid of Formula (8) (manufactured by Sigma-Aldrich Corporation) was dissolved in 32.71 g of a solution of ion-exchanged water-ethanol in a ratio of 1:1 was added to the mixed solution. The reaction was effected for 3 hours and the resultant reaction solution was cooled down to a room temperature. After the completion of the reaction, as by-products, ethanol and methanol were generated.

Thereafter, 200 g of propylene glycol monomethyl ether acetate was added to the reaction solution, and methanol, ethanol and water were distilled off under reduced pressure, followed by warming the reaction mixture at 80° C. for 5 hours to obtain a solution containing a hydrolysis condensate (polymer). The molecular weight of the polymer obtained according to the present Synthetic Example measured by GPC was 5,300 of weight average molecular weight Mw converted into that of polystyrene.

### Synthesis Example 4

In the present Synthetic Example, as an alkoxysilane, tetraethoxysilane, phenyltrimethoxysilane and methyltriethoxysilane (each manufactured by Tokyo Chemical Industry Co., Ltd.) were applied as a raw material monomer.

57.99 g of tetraethoxysilane, 4.25 g of phenyltrimethoxysilane, 22.90 g of methyltriethoxysilane and 85.14 g of ethanol were charged into a 300 mL flask to dissolve them and the resultant mixed solution was warmed and refluxed while stirring the solution with a magnetic stirrer. Next, an aqueous solution in which 1.57 g of hydrochloric acid was dissolved in 28.14 g of ion-exchanged water was added to the mixed solution. The reaction was effected for 2 hours and the resultant reaction solution was cooled down to a room temperature. Instead of hydrochloric acid, another acid acting as a catalyst accelerating the reaction such as nitric acid and phosphoric acid may be used. After the completion of the reaction, as by-products, ethanol and methanol were generated.

Thereafter, 200 g of propylene glycol monomethyl ether acetate was added to the reaction solution, and ethanol, methanol, water and hydrochloric acid were distilled off under reduced pressure to obtain a solution containing a hydrolysis condensate (polymer). In the solvent of the obtained solution, propylene glycol monomethyl ether acetate was contained in a weight ratio close to 100%. The molecular weight of the polymer obtained according to the present Synthetic Example measured by GPC was 7,700 of weight average molecular weight Mw converted into that of polystyrene.

### (Optical Constants)

A composition, which was prepared by adding propylene glycol monomethyl ether to each of the solutions obtained in Synthetic Example 1, Synthetic Example 2 and Synthetic Example 4 to have a concentration of 5% by mass of the solution, was applied onto a silicon wafer using a spinner. Then, the composition was heated at 240° C. for 1 minute to form a resist underlayer film (film thickness: 0.09 μm). Then, the resist underlayer film was subjected to the measurement of a refractive index (n value) and an optical absorptivity (k value, called also as attenuation coefficient) at a wavelength of 193 nm using a spectroscopic ellipsometer (trade name: VUV-VASE VU-302; manufactured by J. A. Woollam Co., Inc.). The measurement results are shown in Table 1.

TABLE 1

| | Refractive | Optical |
|---|---|---|
| | index n | absorptivity k |
| The solution obtained in Synthetic | 1.66 | 0.23 |
| Example 1 was used | | |
| The solution obtained in Synthetic | 1.69 | 0.25 |
| Example 2 was used | | |
| The solution obtained in Synthetic | 1.61 | 0.12 |
| Example 4 was used | | |

### (Dry-Etching Rates)

A composition prepared as described above using each of the solutions obtained in Synthetic Example 1, Synthetic Example 2 and Synthetic Example 4 was applied onto a silicon wafer using a spinner. Then, the composition was heated on a hot plate at 240° C. for 1 minute to form a resist underlayer film (film thickness: 0.09 μm). In addition, in the same manner, a photoresist solution (trade name: UV 113; manufactured by Shipley Corporation) was applied onto a silicon wafer to form an organic resist film.

Thereafter, the dry-etching was performed on the formed resist underlayer film and the organic resist film using CF₄ and O₂ as an etching gas and the dry-etching rate was measured. As the etcher for dry-etching, ES 401 (manufactured by Nippon Scientific Co., Ltd.) for dry-etching with a CF₄ gas and RIE-10NR (manufactured by Samco Inc.) for dry-etching with an O₂ gas were used. Then, the ratio (resist underlayer film/organic resist film) of the dry-etching rate of a resist underlayer film relative to the dry-etching rate of an organic resist film was measured and the results thereof are shown in Table 2.

TABLE 2

| | CF₄ | O₂ |
|---|---|---|
| The solution obtained in Synthetic Example 1 was used | 1.66 | 0.02 |
| The solution obtained in Synthetic Example 2 was used | 1.66 | 0.02 |
| The solution obtained in Synthetic Example 4 was used | 1.66 | 0.02 |

The apparatus and conditions for the measurement of the properties of the compounds used in the following Examples are as follows.

### 1. Mass Spectrometry (MASS)

Apparatus: LX-1000 (trade name; manufactured by JEOL Ltd.)
Detection method: ionization method: DEP (ESI″) m/z=50 to 1,000, DEP (ESI⁺) m/z=50 to 1,000

### 2. ¹H NMR

Apparatus: JNM-LA 400-type FT-NMR system (trade name; manufactured by JEOL Ltd.)
Measuring solvent: DMSO-d₆

### 3. ¹³C NMR

Apparatus: JNM-LA 400-type FT-NMR system (trade name; manufactured by JEOL Ltd.)
Measuring solvent: DMSO-d₆

### 4. Melting Point (mp.)

Measuring equipment: automatic melting point measuring apparatus (trade name: FP 62; manufactured by Mettler-Toledo International Inc.)

### Example 1

To 121.36 g of the solution obtained in Synthetic Example 1 (polymer concentration: 20% by mass), 0.73 g (3% by mass based on the mass of the polymer solid content) of cis-5-norbornene-endo-2,3-dicarboxylic acid of Formula (8) (manufactured by Sigma-Aldrich Corporation), 237.50 g of propylene glycol monomethyl ether and 140.41 g of propylene glycol monomethyl ether acetate were added and the resultant mixture was filtered using a polyethylene microfilter having a pore diameter of 0.05 μm to prepare the resist underlayer film-forming composition of the present invention. The polymer solid content is a component remaining after removing the solvent from the solution, which is the same also in the other Examples and Comparative Examples in the present specification.

### Example 2

To 121.36 g of the solution obtained in Synthetic Example 2 (polymer concentration: 20% by mass), 0.73 g (3% by mass based on the mass of the polymer solid content) of cis-5-norbornene-endo-2,3-dicarboxylic acid of Formula (8) (manufactured by Sigma-Aldrich Corporation), 237.50 g of propylene glycol monomethyl ether and 140.41 g of propylene glycol monomethyl ether acetate were added and the resultant mixture was filtered using a polyethylene microfilter having a pore diameter of 0.05 μm to prepare the resist underlayer film-forming composition of the present invention.

### Example 3

To 121.36 g of the solution obtained in Synthetic Example 1 (polymer concentration: 20% by mass), 0.73 g (3% by mass based on the mass of the polymer solid content) of cis-norbornane-endo-2,3-dicarboxylic acid of Formula (9), 237.50 g of propylene glycol monomethyl ether and 140.41 g of propylene glycol monomethyl ether acetate were added and the resultant mixture was filtered using a polyethylene microfilter having a pore diameter of 0.05 μm to prepare the resist underlayer film-forming composition of the present invention. The synthetic method of the compound of a polycyclic structure of Formula (9) used in the present Example is described as follows.

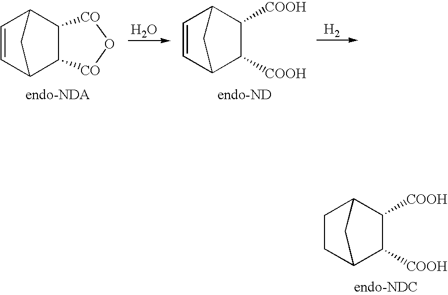

32.8 g (200 mmol) of 5-norbornene-endo-2,3-dicarboxylic acid anhydride available from Sigma-Aldrich Corporation and 100 g of water were charged into a 200 mL four-neck reaction flask and the resultant mixture was warmed and stirred in an oil bath of 120° C. The reaction product was converted from slurry to a homogeneous substance and the reaction was terminated after the reflux of 2 hours. Next, a crystal crystallized by ice-cooling the reaction mixture was washed with water and was reduced pressure-dried to obtain 33.8 g of a white crystal (endo-ND) (yield: 92.8%). The obtained crystal was subjected to the mass spectrometry and the measurement of melting point (melting point: 177.1° C.).

Next, into a 200 mL four-neck reaction flask, 10.1 g (55.4 mmol) of the endo-ND, 100 g of ethanol and 2.2 g of 5% Pd/C (trade name: BNA-SD; manufactured by N.E. Chemcat Corporation; having a water content of 54.54%) as a catalyst were charged. The inside of the flask was nitrogen-purged and thereto, hydrogen was introduced from a balloon, followed by stirring the reaction mixture at 25° C. for 22 hours. The reaction mixture was filtered with a 1 μm filtration paper and the resultant filtrate was concentrated to obtain 10.1 g of a white crystal (yield: 99%).

The obtained crystal was confirmed to be cis-norbornane-endo-2,3-dicarboxylic acid from the results of the mass spectrometry, the measurements by ¹H NMR and ¹³C NMR and the measurement of melting point (152.9° C.) of the crystal.

### Example 4

To 118.34 g of the solution obtained in Synthetic Example 1 (polymer concentration: 20% by mass), 1.33 g (5.63% by mass based on the mass of the polymer solid content) of 2,3-dibromo-endo-5,6-dicarboxynorbornane of Formula (10), 237.50 g of propylene glycol monomethyl ether and 142.83 g of propylene glycol monomethyl ether acetate were added and the resultant mixture was filtered using a polyethylene microfilter having a pore diameter of 0.05 μm to prepare the resist underlayer film-forming composition of the present invention. The synthetic method of the compound of a polycyclic structure of Formula (10) used in the present Example is described as follows.

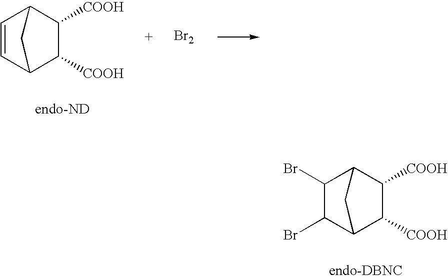

5.47 g (30 mmol) of 5-norbornene-endo-2,3-dicarboxylic acid (endo-ND) and 55 g of 1,2-dichloroethane were charged into a 200 mL four-neck reaction flask and into the resultant reaction mixture, a mixture of 5.28 g (33 mmol) of bromine and 6 g of 1,2-dichloroethane was dropped at 5° C. over 10 minutes while stirring the reaction mixture. Next, while gradually elevating the temperature of the reaction mixture to 25° C., the reaction mixture was stirred further for 1 hour. Thereafter, the reaction mixture was ice-cooled and filtered to obtain a cake and the obtained cake was washed with 16 g of 1,2-dichloroethane and was reduced pressure-dried at 70° C. for 3 hours to obtain 6.58 g of a white crystal (yield; 64.1%).

The obtained crystal was confirmed to be 2,3-dibromo-endo-5,6-dicarboxynorbornane from the results of the mass spectrometry, the measurements by ¹H NMR and ¹³C NMR and the measurement of melting point (209.4° C.) of the crystal.

### Example 5

To 120.55 g of the solution obtained in Synthetic Example 1 (polymer concentration: 20% by mass), 0.89 g (3.7% by mass based on the mass of the polymer solid content) of tricyclo[5.2.1.0^{2,6}]decane-8,9-dicarboxylic acid of Formula (12), 237.50 g of propylene glycol monomethyl ether and 141.06 g of propylene glycol monomethyl ether acetate were added and the resultant mixture was filtered using a polyethylene microfilter having a pore diameter of 0.05 μm to prepare the resist underlayer film-forming composition of the present invention.

The compound of a polycyclic structure of Formula (12) used in the present Example was synthesized by a method described in Japanese Patent Application Publication No, JP-A-7-053453.

### Example 6

To 121.36 g of the solution obtained in Synthetic Example 1 (polymer concentration: 20% by mass), 0.73 g (3% by mass based on the mass of the polymer solid content) of dimethyl-7,8-dicarboxytricyclo[4.2.1.0^{2,5}]nona-3-ene-3,4-dicarboxylate of Formula (11), 237.50 g of propylene glycol monomethyl ether, 92.91 g of propylene glycol monomethyl ether acetate and 47.5 g of γ-butyrolactone were added and the resultant mixture was filtered using a polyethylene microfilter having a pore diameter of 0.05 μm to prepare the resist underlayer film-forming composition of the present invention.

The compound of a polycyclic structure of Formula (11) used in the present Example was synthesized by a method described in Japanese Patent Application Publication No. JP-A-2003-137843.

### Example 7

To 120.28 g of the solution obtained in Synthetic Example 1 (polymer concentration: 20% by mass), 0.94 g (3.92% by mass based on the mass of the polymer solid content) of 3,4-epoxytricyclo[5.2.1.0^{2,6}]decane-8,9-dicarboxylic acid of Formula (13), 237.50 g of propylene glycol monomethyl ether and 141.27 g of propylene glycol monomethyl ether acetate were added and the resultant mixture was filtered using a polyethylene microfilter having a pore diameter of 0.05 μm to prepare the resist underlayer film-forming composition of the present invention.

The compound of a polycyclic structure of Formula (13) used in the present Example was synthesized by a method described in Examined Japanese Patent Application Publication No. JP-B-5-017227.

### Example 8

To 119.82 g of the solution obtained in Synthetic Example 1 (polymer concentration: 20% by mass), 1.04 g (4.32% by mass based on the mass of the polymer solid content) of 3,4-epoxytetracyclo[5.4.1.0^{2,6}.0^{8,11}]dodeca-9-ene-9,10-dicarboxylic acid of Formula (14), 237.50 g of propylene glycol monomethyl ether and 141.64 g of propylene glycol monomethyl ether acetate were added and the resultant mixture was filtered using a polyethylene microfilter having a pore diameter of 0.05 μm to prepare the resist underlayer film-forming composition of the present invention. The synthetic method of the compound of a polycyclic structure of Formula (14) used in the present Example is described as follows.

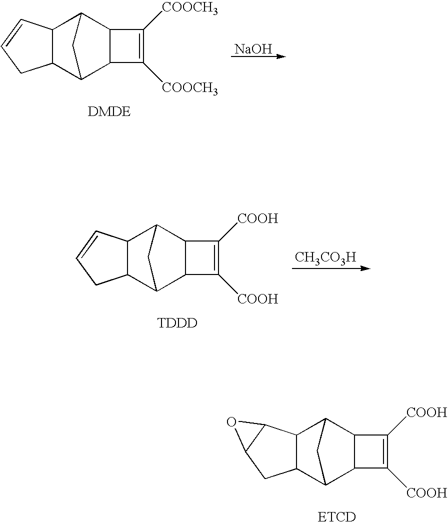

27.4 g (100 mmol) of dimethyltetracyclo[5.4.1.0^{2,6}0^{8,11}]dodeca-3,9-diene-9,10-dicarboxylate (DMDE) synthesized by a method described in Japanese Patent Application Publication No. JP-A-9-077721 and Japanese Patent Application Publication No. JP-A-2004-224748 and 87 g of methanol were charged into a 500 mL four-neck reaction flask and into the resultant reaction mixture, a solution in which 12 g (300 mmol) of sodium hydroxide was dissolved in 72 g of water was dropped at 5° C. while stirring the reaction mixture. Next, while elevating the temperature of the reaction mixture, the reaction mixture was stirred for 9 hours in an oil bath of 100° C. (inside temperature: 72° C.). Methanol was concentrated and into the resultant residue, which was being ice-cooled, 30 g of 35% hydrochloric acid was dropped to acidify the resultant reaction mixture, so that a block crystal was separated out. When the reaction mixture was stirred for 3 hours, the block crystal became slurry and by subjecting the slurry to filtration, water-washing and reduced pressure-drying, 24.7 g of a skin-colored crystal was obtained. Further, 68 g of 1,4-dioxane was added to the crystal and while heating the resultant mixture to 70° C., the crystal was dissolved, followed by filtering the resultant solution while heating the solution. The resultant filtrate was concentrated and thereto, acetonitrile was added, followed by heating the resultant mixture to 70° C. to convert the mixture to slurry. Thereafter, the slurry was left stand still at 25° C. overnight and the slurry was solidified, so that the filtrate was concentrated. Thereafter, to the concentrated filtrate, ethyl acetate was added to convert the filtrate to slurry and the slurry was filtered. The resultant filtrate was washed with a solvent mixture of ethyl acetate/n-heptane=1/3 and was reduced pressure-dried to obtain 17.8 g of a white crystal (yield: 72.3%). The obtained crystal was confirmed to be tetracyclo[5.4.1.0^{2,6}.0^{8,11}]dodeca-3,9-diene-9,10-dicarboxylic acid (TDDD) from the results of the mass spectrometry and the melting point (235.9° C.) measurement of the crystal.

4.5 g (18 mmol) of TDDD, 25 g of 1,4-dioxane and 1.0 g of di-sodium hydrogen phosphate were charged into a 100 mL four-neck flask and into the resultant reaction mixture, 6.9 g (36 mmol) of 40% peracetic acid was dropped at 5° C. while stirring the reaction mixture. Next, the reaction mixture was heated to 24° C. and was stirred for 24 hours. The reaction mixture was concentrated and to the resultant residue, ethyl acetate and water were added to separate the organic layer. The organic layer was washed with water and was reduced pressure-dried to obtain 2.4 g of a crystal (yield: 50.8%).

The obtained crystal was confirmed to be 3,4-epoxytetracyclo[5.4.1.0^{2,6}.0^{8,11}]dodeca-9-ene-9,10-dicarboxylic acid from the results of the mass spectrometry and the melting point (235,9° C.) measurement of the crystal.

### Example 9

To 32.82 g of the solution containing cis-5-norbornene-endo-2,3-dicarboxylic acid and a polymer that was obtained in Synthetic Example 3, 9.88 g of propylene glycol monomethyl ether acetate, 9.55 g of propylene glycol monomethyl ether and 47.75 g of propylene glycol monopropyl ether were added and the resultant mixture was filtered using a polyethylene microfilter having a pore diameter of 0.05 μm to prepare the resist underlayer film-forming composition of the present invention.

### Example 10

To 121.36 g of the solution obtained in Synthetic Example 1 (polymer concentration: 20% by mass), 0.73 g (3% by mass based on the mass of the polymer solid content) of 2,3-naphthalenedicarboxylic acid of Formula (15) (manufactured by Wako Pure Chemical Industries Co., Ltd.), 237.50 g of propylene glycol monomethyl ether and 140.41 g of propylene glycol monomethyl ether acetate were added and the resultant mixture was filtered using a polyethylene microfilter having a pore diameter of 0.05 μm to prepare the resist underlayer film-forming composition of the present invention.

### Example 11

To 142.04 g of the solution obtained in Synthetic Example 4, 0.06 g of benzyltriethylammonium chloride, 0.95 g of cis-5-norbornene-endo-2,3-dicarboxylic acid of Formula (8) (manufactured by Sigma-Aldrich Corporation), 240.0 g of propylene glycol monopropyl ether, 26.35 g of propylene glycol monomethyl ether acetate and 96.00 g of propylene glycol monomethyl ether were added and the resultant mixture was filtered using a polyethylene microfilter having a pore diameter of 0.05 μm to prepare the resist underlayer film-forming composition of the present invention.

### Comparative Example 1

To 125.00 g of the solution obtained in Synthetic Example 1 (polymer concentration: 20% by mass), 237.50 g of propylene glycol monomethyl ether and 137.50 g of propylene glycol monomethyl ether acetate were added and the resultant mixture was filtered using a polyethylene microfilter having a pore diameter of 0.05 μm to prepare a resist underlayer film-forming composition.

The resist underlayer film-forming composition of the present Comparative Example is different from those of the above Examples in terms of containing no compound of a polycyclic structure having at least two carboxyl groups as substituents.

### Comparative Example 2

To 121.36 g of the solution obtained in Synthetic Example 1 (polymer concentration: 20% by mass), 0.73 g (3% by mass based on the mass of the polymer solid content) of phthalic acid of Formula (16) (manufactured by Tokyo Chemical Industry Co., Ltd.), 237.50 g of propylene glycol monomethyl ether and 140.41 g of propylene glycol monomethyl ether acetate were added and the resultant mixture was filtered using a polyethylene microfilter having a pore diameter of 0.05 μm to prepare a resist underlayer film-forming composition.

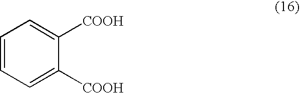

Phthalic acid used in the present Comparative Example is a dicarboxylic acid, however, it is apparently not a compound of a polycyclic structure.

### Comparative Example 3

To 121.36 g of the solution obtained in Synthetic Example 1 (polymer concentration: 20% by mass), 0.73 g (3% by mass based on the mass of the polymer solid content) of 5-norbornene-2-endo,3-exo-dicarboxylic acid of Formula (17) (manufactured by Sigma-Aldrich Corporation), 237.50 g of propylene glycol monomethyl ether and 140.41 g of propylene glycol monomethyl ether acetate were added and the resultant mixture was filtered using a polyethylene microfilter having a pore diameter of 0.05 μm to prepare a resist underlayer film-forming composition.

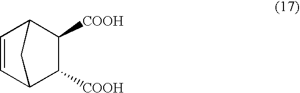

5-norbornene-2-endo,3-exo-dicarboxylic acid used in the present Comparative Example is a compound of a polycyclic structure having two carboxyl groups as substituents and the two carboxyl groups are individually bonded to two carbon atoms adjacent to each other forming the polycyclic structure, however, the two carboxyl groups are not arranged adjacent to each other. In other words, the two carboxyl groups have a configuration in which one of them has the endo configuration and the other has the exo configuration.

### (Evaluation of Solvent Resistance)

Each of the resist underlayer film-forming compositions of Example 1 to Example 11 and Comparative Example 1 to Comparative Example 3 of the present specification was applied onto a silicon wafer by a spin coating method and the composition was heated on a hot plate at 240° C. for 1 minute to form a resist underlayer film (layer B) containing silicon atoms. Thereafter, the resist underlayer film was immersed in propylene glycol monomethyl ether acetate for 1 minute and the change in the film thickness of the resist underlayer film between before and after the immersion was measured. As the result of the measurement, the change in the film thickness was found to be 2 nm or less with respect to every resist underlayer film.

### (Evaluation of Developer Resistance)

Each of the resist underlayer film-forming compositions of Example 1 to Example 11 and Comparative Example 1 to Comparative Example 3 of the present specification was applied onto a silicon wafer by a spin coating method and the composition was heated on a hot plate at 240° C. for 1 minute to form a resist underlayer film (layer B) containing silicon atoms. Thereafter, the resist underlayer film was immersed in a tetramethylammonium hydroxide aqueous solution of 2.38% by mass for 1 minute and the change in the film thickness of the resist underlayer film between before and after the immersion was measured. As the result of the measurement, the change in the film thickness was found to be 2 nm or less with respect to every resist underlayer film.

### (Evaluation of Resist Patterning)

30 g of 2-vinylnaphthalene, 3.5 g of glycidylmethacrylate and 4.5 g of 1-butoxyethylmethacrylate were dissolved in 112 g of cyclohexanone and the inside of the flask was nitrogen-purged and was heated to 60° C. After the heating, to the resultant reaction mixture, 1.9 g of azobisisobutylonitrile dissolved in 48 g of cyclohexanone was added under nitrogen pressure and the reaction was effected at 60° C. for 24 hours. The reaction solution was cooled down and was introduced into methanol to re-precipitate a polymer and the polymer was heating-dried to obtain a polymer of the following Formula (18). The molecular weight measured by GPC of the obtained polymer was 12,000 of the weight average molecular weight Mw converted into that of polystyrene. In Formula (18), when the ratio of the total repeating unit is assumed to be 1.0 (100 mol %), the ratio of a repeating unit containing 2-vinylnaphthalene is 0.8 (80 mol %); the ratio of a repeating unit containing 1-butoxyethylmethacrylate is 0.1 (10 mol %); and the ratio of a repeating unit containing glycidylmethacrylate is 0.1 (10 mol %).

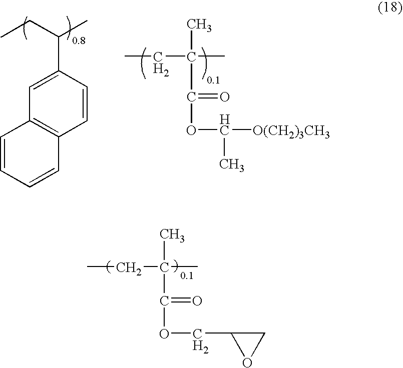

5 g of the obtained polymer was mixed with 0.03 g of a surfactant (trade name: MEGAFAC R-30; manufactured by DIC Corporation) and the resultant mixture was dissolved in 23 g of cyclohexanone and 23 g of propylene glycol monomethyl ether to prepare a solution. Thereafter, the solution was filtered using a polyethylene microfilter having a pore diameter of 0.10 μm and further using a polyethylene microfilter having a pore diameter of 0.05 μm to prepare a resist underlayer film-forming composition used in a lithography process. A resist underlayer film (layer A) formed from the composition and containing no silicon resin was combined with a resist underlayer film (layer B) formed from each of the resist underlayer film-forming compositions of Example 1 to Example 11 and Comparative Example 1 to Comparative Example 3 of the present specification to constitute a multilayer film.

A resist underlayer film-forming composition containing a polymer of Formula (18) was applied onto a silicon wafer and the composition was heated on a hot plate at 240° C. for 1 minute to form a resist underlayer film (layer A) having a film thickness of 250 nm. Onto the layer A, each of the resist underlayer film-forming compositions of Example 1 to Example 11 and Comparative Example 1 to Comparative Example 3 was applied by a spin coating method and the composition was heated on a hot plate at 240° C. for 1 minute to form a resist underlayer film (layer B) having a film thickness of 80 nm. Onto the layer B, a commercially available photoresist solution (trade name: PAR 855; manufactured by Sumitomo Chemical Company Limited) was applied by a spinner and the solution was heated on a hot plate at 100° C. for 1 minute to form a photoresist film (layer C) having a film thickness of 150 nm.

The patterning of the resist was performed using a scanner (trade name: PAS 5500/1100; manufactured by ASML Corporation; wavelength: 193 nm, NA, a: 0.75, 0.89/0.59 (Dipole)). The target was a resist pattern after the development having a line width and a width between lines both of 0.08 μm, which is a so-called “line and space (dense line) pattern”, and the exposure was performed through a photomask set so that 9 lines were formed. Thereafter, the resist pattern was heated on a hot plate at 105° C. for 1 minute, was cooled down, and was developed by a 60 second single paddle-type process according to JIS using a developer (2.38% by mass tetramethylammonium hydroxide aqueous solution).

The focus depth margin was determined as follows. That is, the above exposure was performed while displacing the position of the focus upward and downward by 0.1 μm on a basis of the position of the optimal focus and the development treatment was performed to form a resist pattern. Then, when among the 9 lines of the resist pattern to be formed, 5 or more lines were formed without being collapsed, the resist pattern was evaluated as qualified by the test. On the contrary, when the number of remaining lines was less than 5, the resist pattern was evaluated as not qualified by the test. Then, the displacing depth of the focus position between the uppermost and the lowermost capable of obtaining the result of “qualified” was regarded as a focus depth margin. Therefore, when the resist pattern was not qualified, there existed no value of focus depth margin.

TABLE 3

| | Focus depth margin | |
|---|---|---|
| | (μm) | Line bottom shape |
| Example 1 | 0.7 | Advantageous |
| Example 2 | 0.6 | Advantageous |
| Example 3 | 0.5 | Advantageous |
| Example 4 | 0.5 | Advantageous |
| Example 5 | 0.5 | Advantageous |
| Example 6 | 0.5 | Advantageous |
| Example 7 | 0.5 | Advantageous |
| Example 8 | 0.5 | Advantageous |
| Example 9 | 0.6 | Advantageous |
| Example 10 | 0.5 | Advantageous |
| Example 11 | 0.7 | Advantageous |
| Comparative Example 1 | — | Pattern collapse |
| Comparative Example 2 | — | Pattern collapse |
| Comparative Example 3 | — | Pattern collapse |

Table 3 shows the focus depth margin and the bottom shape of the resist pattern line of each of Examples and Comparative Examples of the present specification. In Table 3, the line bottom shape shows the result of observing the top face shape of the resist pattern and the cross section shape of the resist pattern in a direction perpendicular to the substrate, and each line preferably has a substantially rectangle shape. From the viewpoint of the focus depth margin, Example 1 and Example 11 were the most preferred, and Example 2 and Example 9 were secondly preferred. On the contrary, in Comparative Example 1 to Comparative Example 3, a collapse of the resist pattern to be formed was observed. Thus, it has been demonstrated that the compound of a polycyclic structure that has at least two carboxyl groups as substituents in which the two carboxyl groups are individually bonded to two carbon atoms adjacent to each other forming the polycyclic structure, and the two carboxyl groups both have an endo configuration or an exo configuration, or have a cis configuration, and that was used in each Example of the present specification, is useful as an additive for the resist underlayer film-forming composition of the present invention.

### (Production of Semiconductor Element)

As described above, on a silicon wafer, a resist underlayer film (layer A), a resist underlayer film (layer B) and a photoresist film (layer C) are formed in this order and the resultant laminate is subjected to exposure using a scanner, post exposure bake and development to form a resist pattern. The formed resist pattern has a substantially rectangle shape of each line without being collapsed.

Then, using the formed resist pattern as a mask, the dry-etching is performed relative to the resist underlayer film (layer B) using a gas containing CF₄ to form a pattern of the resist underlayer film (layer B). Using the pattern of the resist underlayer film (layer B) and the resist pattern as masks, the dry-etching is performed relative to the resist underlayer film (layer A) on the silicon wafer using a gas containing O₂ to form a pattern of the resist underlayer film (layer A). At this time, the resist pattern is removed.

Continuously, through a process such as processing the silicon wafer by a publicly known technique, a semiconductor element can be produced. Here, in the finally produced semiconductor device, the pattern of the resist underlayer film is removed. As described above, the resist underlayer film-forming composition of the present invention can be used in a lithography process during the production of a semiconductor element.

1. A resist underlayer film-forming composition for lithography comprising:
a polymer having silicon atoms in the backbone;
a compound of a polycyclic structure; and
an organic solvent, wherein
the compound of a polycyclic structure has at least two carboxyl groups as substituents;
the two carboxyl groups are individually bonded to two carbon atoms adjacent to each other forming the polycyclic structure; and
the two carboxyl groups both have an endo configuration or an exo configuration, or have a cis configuration.

2. The resist underlayer film-forming composition for lithography according to claim 1, wherein
the compound of a polycyclic structure is a compound of Formula (1): 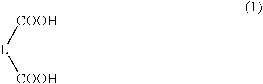
(where L is derived from a hydrocarbon of a polycyclic structure of Formula (2), Formula (3), Formula (4), Formula (5), Formula (6) or Formula (7): 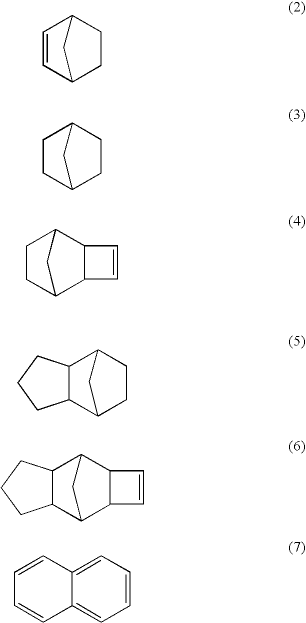
and is a divalent group in which two carboxyl groups are bonded to two carbon atoms adjacent to each other forming the polycyclic structure; and
the hydrocarbon of a polycyclic structure of Formula (2) to Formula (7) optionally has a substituent and/or is optionally epoxidized).

3. The resist underlayer film-forming composition for lithography according to claim 2, wherein at least one hydrogen atom in the hydrocarbon of a polycyclic structure of Formula (2) to Formula (7) is replaced by a halogen atom.

4. The resist underlayer film-forming composition for lithography according to claim 1, wherein the compound of a polycyclic structure is an alicyclic hydrocarbon having a bicyclo ring, a tricyclo ring or a tetracyclo ring.

5. The resist underlayer film-forming composition for lithography according to claim 1, wherein the compound of a polycyclic structure is an alicyclic dicarboxylic acid of a polycyclic structure.

6. The resist underlayer film-forming composition for lithography according to claim 1, wherein the compound of a polycyclic structure is 3,4-epoxytetracyclo[5.4.1.0^{2,6}.0^{8,11}]dodeca-9-ene-9,10-dicarboxylic acid.

7. The resist underlayer film-forming composition for lithography according to claim 1, wherein the polymer having silicon atoms in the backbone is a hydrolysis condensate of at least two types of alkoxysilanes.

8. An additive for a resist underlayer film-forming composition comprising a compound of a polycyclic structure having at least two carboxyl groups as substituents, wherein
the two carboxyl groups are individually bonded to two carbon atoms adjacent to each other forming the polycyclic structure; and
the two carboxyl groups both have an endo configuration or an exo configuration, or have a cis configuration.

9. The additive for a resist underlayer film-forming composition according to claim 8, wherein the compound of a polycyclic structure is 3,4-epoxytetracyclo[5.4.1.0^{2,6}.0^{8,11}]dodeca-9-ene-9,10-dicarboxylic acid.
